(19) United States
(12) Patent Application Publication
(10) Pub. No.: US 2014/0228670 A1
(43) Pub. Date: Aug. 14, 2014

(54) SURGICAL IMPLANT GUIDE SYSTEM AND METHOD

(71) Applicant: WARSAW ORTHOPEDIC, INC., Warsaw, IN (US)

(72) Inventors: Jeff R. Justis, Germantown, TN (US); Richard L. Brown, Mesa, AZ (US)

(73) Assignee: Warsaw Orthopedic, Inc., Warsaw, IN (US)

(21) Appl. No.: 13/764,868

(22) Filed: Feb. 12, 2013

(51) Int. Cl. A61B 5/06; A61B 17/70; A61B 5/00

(52) U.S. Cl. CPC A61B 5/062; A61B 5/0002; A61B 5/686; A61B 5/742; A61B 17/7082
USPC 600/409

(57) ABSTRACT

A surgical system comprises a magnet having a magnetic field. The magnet is disposed with a first spinal implant. A sensor is disposed with a first end of a second spinal implant. The sensor is configured to measure the magnetic field relative to the second spinal implant. Indicia represents position and orientation of the first spinal implant relative to the second spinal implant based on the measured magnetic field. Systems and methods are disclosed.

### TECHNICAL FIELD

The present disclosure generally relates to medical devices for the treatment of spinal disorders, and more particularly to a spinal implant system for guiding an implant and a method for treating a spine.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes discectomy, laminectomy, fusion, correction and implantable prosthetics. As part of these surgical treatments, spinal constructs such as bone fasteners and vertebral rods are often used to provide stability to a treated region. For example, during surgical treatment, surgical instruments can be used to deliver and/or introduce the spinal constructs adjacent to a surgical site for fixation with bone to immobilize a joint. This disclosure describes an improvement over these prior art technologies.

### SUMMARY

In one embodiment, in accordance with the principles of the present disclosure, a spinal implant guide is provided. The spinal implant guide includes a magnet having a magnetic field disposed with a first spinal implant. A sensor is disposed with a first end of a second spinal implant. The sensor is configured to measure the magnetic field relative to the second spinal implant. Indicia represents position and orientation of the first spinal implant relative to the second spinal implant based on the measured magnetic field.

### BRIEF DESCRIPTION OF THE DRAWINGS

The present disclosure will become more readily apparent from the specific description accompanied by the following drawings, in which:

FIG. 1 is a perspective view of components of one embodiment of a spinal implant system in accordance with the principles of the present disclosure;

FIG. 2 is a perspective view of components of the system shown in FIG. 1;

FIG. 3 is a perspective view of the components shown in FIG. 2 with parts separated;

FIG. 4 is a perspective view of components of the system shown in FIG. 1;

FIG. 5 is a perspective view of components of a spinal implant system in accordance with the principles of the present disclosure disposed with a body; and

FIG. 6 is a plan view of components of the system shown in FIG. 5 disposed with the body.

Like reference numerals indicate similar parts throughout the figures.

### DETAILED DESCRIPTION

The exemplary embodiments of a surgical system are discussed in terms of medical devices for the treatment of musculoskeletal disorders and more particularly, in terms of a spinal implant system for guiding an implant to a desired location, and a method for implementing the spinal implant system. In one embodiment, the system is employed in a minimally invasive procedure to guide a spinal rod into a saddle of a spinal screw. This configuration avoids undesired engagement with tissue, such as, for example, pinching of tissue between the rod and the saddle.

In one embodiment, the system includes an electronic guide that includes a guidance tool configured to engage a first implant such as, for example, a fastener. In one embodiment, the guidance tool is configured to slip over the top of the fastener. In one embodiment, the system includes a 3-axis magnetometer disposed adjacent the saddle of a screw. In one embodiment, a magnet is positioned on a tip of the rod such that the magnetometer can measure the position of the magnet to guide the rod into a saddle of the fastener. This configuration reduces the time for a spinal procedure while facilitating selective placement of the rod in the saddle of the fastener.

In one embodiment, the guidance tool may be removed from the fastener after the rod is positioned within the saddle, according to the preference of a medical practitioner. In one embodiment, the guidance tool is a temporary slip-on device such that the magnet removably engages the rod to allow the magnet to be removed from the rod after the rod is positioned within the saddle.

In one embodiment, information gathered by the guidance tool is presented at the point of use, such as, for example, in an operation room. In one embodiment, the information gathered by the guidance tool may be provided using a display that is mounted on the guidance tool. In one embodiment, the guidance tool can include a wireless radio frequency link to transmit the information gathered by the guidance tool to another device such as, for example, a computer located inside or outside the operating room. In one embodiment, the guidance tool can be hardwired to another device such as, for example, a computer.

It is envisioned that the system of the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the system of the present disclosure may be employed with other osteal and bone related applications, including those associated with diagnostics and therapeutics. In some embodiments, the disclosed system may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The system of the present disclosure may also be alternatively employed with procedures for treating the lumbar, cervical, thoracic, sacral and pelvic regions of a spinal column. The system of the present disclosure may also be used on animals, bone models and other non-living substrates, such as, for example, in training, testing and demonstration.

The system of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

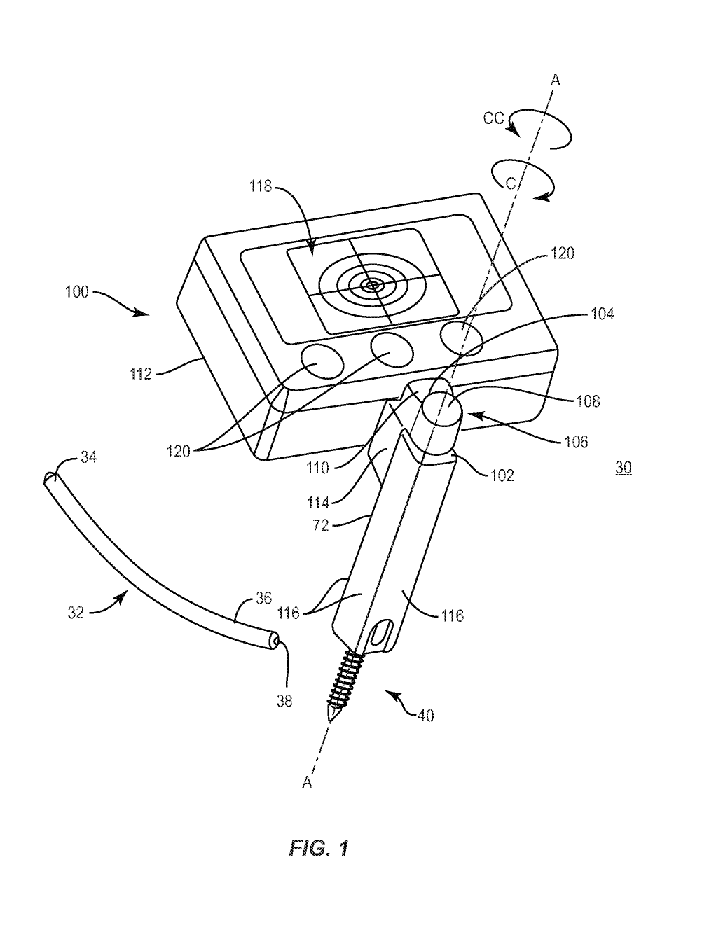

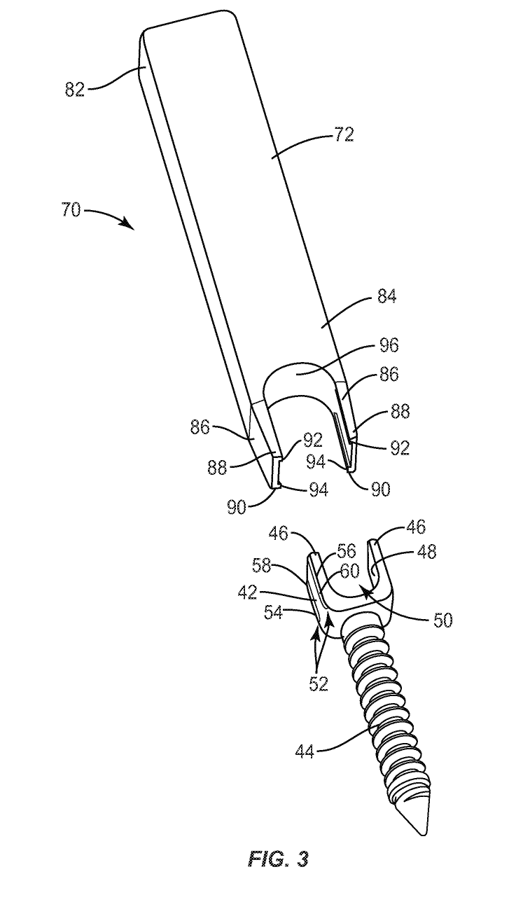

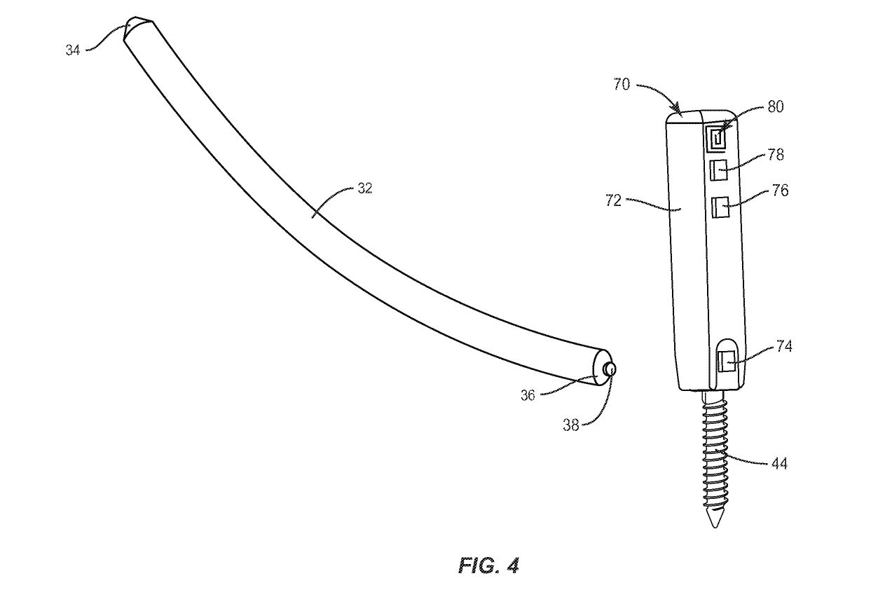

The following discussion includes a description of a surgical system including a surgical instrument, related components and methods of employing the surgical system in accordance with the principles of the present disclosure. Alternate embodiments are also disclosed. Reference will now be made in detail to the exemplary embodiments of the present disclosure, which are illustrated in the accompanying figures. Turning now to FIGS. 1-4, there are illustrated components of a surgical implant system 30, in accordance with the principles of the present disclosure.

The components of system 30 can be fabricated from biologically acceptable materials suitable for medical applications, including metals, synthetic polymers, ceramics and bone material and/or their composites, depending on the particular application and/or preference of a medical practitioner. For example, the components of system 30, individually or collectively, can be fabricated from materials such as stainless steel alloys, aluminum, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 30 may have material composites, including the above materials, to achieve various desired characteristics such as strength, rigidity, elasticity, compliance, biomechanical performance, durability and radiolucency or imaging preference. The components of system 30, individually or collectively, may also be fabricated from a heterogeneous material such as a combination of two or more of the above-described materials. The components of system 30 may be monolithically formed, integrally connected or include fastening elements and/or instruments, as described herein.

System 30 includes a first spinal implant, such as, for example, a vertebral rod 32. Rod 32 is substantially cylindrical and extends between a first end 34 and a second end 36. Rod 32 includes a device configured to emit a magnetic field such as, for example, a magnet 38 removably disposed on the outer surface of rod 32 at end 34. Magnet 38 emits a magnetic field that is measured by a sensor, such as, for example, a magnetometer 74 positioned adjacent an implant, such as, for example, a fastener 40 to determine the position and orientation of magnet 38 relative to fastener 40 in order to facilitate guidance of rod 32 through the anatomy of a patient to engage rod 32 with fastener 40. In some embodiments, magnet 38 may be disposed on an end face or tip of end 34 that extends perpendicular to an axis defined by rod 32. In some embodiments, magnet 38 may be disposed on end 36, such as, for example, an end face or tip of end 36 that extends perpendicular to an axis defined by rod 32, or at any location along rod 32 between end 34 and end 36. In some embodiments, the magnet may be disposed with rod 32 in alternate fixation configurations, such as, for example, friction fit, pressure fit, locking protrusion/recess, locking keyway and/or adhesive. In some embodiments, all or only a portion of rod 32 and/or magnet 38 may have alternate surface configurations to enhance fixation with the other of rod 32 and magnet 38, such as, for example, rough, arcuate, undulating, mesh, porous, semi-porous, dimpled and/or textured, according to the requirements of a particular application. In one embodiment, magnet 38 is embedded within rod 32. In one embodiment, magnetometer 74 is a three-axis magnetometer. In one embodiment, magnet 38 is an electromagnet, which may be activated remotely to emit a magnetic field.

As magnet 38 moves to a closer proximity and/or away from magnetometer 74, the strength, flux, and direction of the magnetic field emitted by magnet 38 changes and/or can be adjusted with relative movement of the components of system 30. Magnetometer 74 detects these changes. Information gathered by magnetometer 74 relating to changes in the magnetic field of magnet 38 detected by magnetometer 74 are then processed by a processor, such as, for example, a microcontroller 76 that communicates with magnetometer 74 and determines the position and orientation of magnet 38 relative to magnetometer 74, which directly relates to the position and orientation of rod 32 relative to fastener 40, based upon the changes in the magnetic field of magnet 38 detected by magnetometer 74. The distance between magnet 38 and magnetometer 74 for example, is a function of the strength of the magnetic field of magnet 38.

Microcontroller 76 sends information relating to the position and orientation of magnet 38 relative to magnetometer 74 to an indicia, such as, for example, a display 100, that displays a visual representation of the position and orientation of magnet 38 relative to magnetometer 74, based upon the information send from microcontroller 76. In some embodiments, the indicia can include visual indicia, such as, for example, those described herein, and/or audible indicia.

Fastener 40 defines an axis A and extends between a proximal portion, such as, for example, a receiver 42 and a distal portion such as, for example, a shaft 44 configured to penetrate tissue to fix fastener 40 therein. Receiver 42 includes a pair of spaced apart arms 46 extending parallel to axis A. In one embodiment, arms 46 include one or a plurality of recesses 52 extending parallel to axis A and configured to receive a portion of tool 70 to engage fastener 40 in removable fixation therewith.

Arms 46 each extend between a first end 54 and an opposite second end 56. Ends 54 each include a recess 58 and end 56, which each include a recess 60. Recesses 58, 60 extend parallel to axis A along arms 46. In some embodiments, either or both arms 46 may be disposed at alternate orientations, relative to axis A, such as, for example, transverse, perpendicular and/or other angular orientations such as acute or obtuse, co-axial and/or may be offset or staggered. In some embodiments, recesses 52, 58, 60 may be disposed at alternate orientations, relative to axis A, such as, for example, transverse, perpendicular and/or other angular orientations such as acute or obtuse, co-axial and/or may be offset or staggered. In some embodiments, recesses 52, 58, 60 may be variously configured and dimensioned, such as, for example, convex, concave, polygonal, irregular, uniform, non-uniform, staggered, tapered, consistent or variable, depending on the requirements of a particular application. In some embodiments, recesses 52, 58, 60 may have alternate surface configurations to enhance fixation with tool 70, such as, for example, rough, arcuate, undulating, mesh, porous, semi-porous, dimpled and/or textured according to the requirements of a particular application.

An inner surface 48 of bone fastener 40 defines a U-shaped cavity 50 extending between arms 46. In some embodiments, all or only a portion of cavity 50 may have alternate cross section configurations, such as, for example, oval, oblong, triangular, square, polygonal, irregular, uniform, non-uniform, offset, staggered, and/or tapered. A portion of inner surface 48 may be threaded and engageable with a coupling member, such as, for example, a setscrew. In some embodiments, surface 48 can include a thread form located adjacent arms 46 configured for engagement with a setscrew. In some embodiments, surface 48 may be disposed with the setscrew in alternate fixation configurations, such as, for example, friction fit, pressure fit, locking protrusion/recess, locking keyway and/or adhesive. In some embodiments, all or only a portion of surface 48 may have alternate surface configurations to enhance fixation with the setscrew such as, for example, rough, arcuate, undulating, mesh, porous, semi-porous, dimpled and/or textured according to the requirements of a particular application.

In one embodiment, rod 32 is disposed to extend along an axial plane, such as for example, a sagittal plane of a body of a patient. In some embodiments, system 30 may include one or a plurality of rods 32. In some embodiments, rod(s) 32 may be disposed in various relative orientations, such as, for example, side-by-side, parallel, transverse, perpendicular or angular and/or be disposed to extend along a coronal, sagittal and transverse planes of the body and geometric variations thereof. It is envisioned that rod 32 may have various cross section configurations, such as, for example, oval, oblong, triangular, rectangular, square, polygonal, irregular, uniform, non-uniform, variable and/or tapered.

Rod 32 has a smooth or even outer surface defining a uniform thickness. In some embodiments, rod 32 may have various surface configurations, such as, for example, rough, threaded for connection with surgical instruments, arcuate, undulating, porous, semi-porous, dimpled, polished and/or textured according to the requirements of a particular application. Rod 32 has a length defined by the distance between ends 34, 36. In some embodiments, the thickness defined by the outer surface of rod 32 may be uniformly increasing or decreasing, or have alternate thicknesses along its length.

In some embodiments, rod 32 may have various lengths, according to the requirements of a particular application. In one embodiment, the spinal implant may include a longitudinal element comprising a tether, which may be braided, such as a rope, or include a plurality of elongated elements to provide a predetermined force resistance. In some embodiments, rod 32 may be made from autograft and/or allograft and be configured for resorbable or degradable applications.

In some embodiments, rod 32 may be rigid or semi-rigid and may be constructed from metals, including for example stainless steel, cobalt-chrome, titanium, and shape memory alloys. In some embodiments, rod 32 may be straight, curved, or comprise one or more curved portions along its length. In some embodiments, all or only a portion of rod 32 may have a flexible or elastic configuration and/or have elastic and/or flexible properties, similar to the properties from materials, such as, for example, fabric, silicone, polyurethane, silicone-polyurethane, copolymers, rubbers, polyolefin rubber, elastomers, thermoplastic elastomers, thermoset elastomers and elastomeric composites. In one embodiment, rod 32 provides a selective amount of expansion and/or extension in an axial direction. It is contemplated that rod 32 may have a flexible configuration, which includes movement in a lateral or side to side direction. It is further contemplated that rod 32 may be compressible in an axial direction. Rod 32 can include a plurality of separately attachable or connectable portions or sections, such as bands or loops, or may be monolithically formed as a single continuous element.

Fastener 40 may be employed as a bone screw, pedicle screw or multi-axial screw used in spinal surgery. In one embodiment, the spinal implant system includes an agent, which may be disposed, packed or layered within, on or about the surfaces of fastener 40. It is envisioned that the agent may include bone growth promoting material, such as, for example, bone graft to enhance fixation of the fixation elements with vertebrae.

In some embodiments, the agent may include therapeutic polynucleotides or polypeptides. In some embodiments, the agent may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines. The components of the spinal implant system can be made of radiolucent materials such as polymers. Radiomarkers may be included for identification under x-ray, fluoroscopy, CT or other imaging techniques. In some embodiments, the agent may include one or a plurality of therapeutic agents and/or pharmacological agents for release, including sustained release, to treat, for example, pain, inflammation and degeneration.

Tool 70 is configured to engage fastener 40 and includes a housing 72 configured for disposal of components, such as, for example, magnetometer 74 and microcontroller 76. Housing 72 is configured for disposal of other components, such as, for example, a transmitter 78 and an antenna 80 in communication with transmitter 78. In one embodiment, magnetometer 74 is positioned adjacent cavity 50 when tool 70 engages fastener 40. In one embodiment, housing 72 has a substantially rectangular configuration extending from a first proximal end 82 to a second distal end 84.

Transmitter 78 relays data regarding spatial orientation and location of rod 32, relative to fastener 40, from microcontroller 76. Housing 72 may include one or a plurality of recesses configured for disposal of components, such as, for example, magnetometer 74, microcontroller 76, transmitter 78 and antenna 80. Housing 72 may also be hollow, such that housing 72 includes an inner surface defining a passageway configured for disposal of components, such as, for example, magnetometer 74, microcontroller 76, transmitter 78 and antenna 80. In some embodiments, at least one of magnetometer 74, microcontroller 76, transmitter 78 and antenna 80 may be embedded within housing 72. In some embodiments, all or only a portion of housing 72 may be variously configured and dimensioned, such as, for example, oval, oblong, square, rectangular, polygonal, irregular, uniform, non-uniform, offset, staggered, tapered, consistent or variable, depending on the requirements of a particular application.

End 84 includes a pair of spaced apart arms 86 each extending parallel to axis A. Arms 86 each extend from a first end 88 to an opposite second end 90. Ends 88 each include a flange 92 extending transverse to axis A along arm 86 configured for disposal of in a respective recess 58. Ends 90 each include a flange 94 extending transverse to axis A along arm 86 configured for disposal in a respective recess 60. This configuration allows a medical practitioner to engage tool 70 with fastener 40 by positioning tool 70 relative to fastener 40 such that flanges 92, 94 are aligned with recesses 58, 60 and translating tool 70 axially, in the direction shown by arrow A. As tool 70 is translated axially relative to fastener 40, in the direction shown by arrow A, flanges 92, 94 are disposed within recesses 58, 60 to engage fastener 40 with tool 70. This configuration prevents rotation of tool 70 relative to fastener 40 about axis A, in the direction shown by arrow C in FIG. 1, or, in the direction shown by arrow CC. In some embodiments, flanges 92, 94 may be disposed at alternate orientations, relative to first longitudinal axis a, such as, for example, transverse, perpendicular and/or other angular orientations such as acute or obtuse, co-axial and/or may be offset or staggered. In some embodiments, flanges 92, 94 may be variously configured and dimensioned, such as, for example, convex, concave, polygonal, irregular, uniform, non-uniform, staggered, tapered, consistent or variable, to facilitate engaging disposal of flanges 92, 94 in recesses 58, 60. In some embodiments, flanges 92, 94 may have alternate surface configurations to enhance engagement of tool 70 with fastener 40 such as, for example, rough, arcuate, undulating, mesh, porous, semi-porous, dimpled and/or textured. In some embodiments, tool 70 may engage fastener 40 in various fixation configurations, such as, for example, snap fit, friction fit, pressure fit, clips and/or adhesive.

Arms 86 define an arcuate portion 96 of housing 72 extending between arms 86. Cavity 50 and portion 96 define an oblong channel 98 configured to assist a medical practitioner in guiding rod 32 into cavity 50, as will be discussed. In one embodiment, magnetometer 74 is positioned adjacent portion 96. In one embodiment, magnetometer 74 is embedded within portion 96. In some embodiments, portion 96 and/or channel 98 may be variously configured and dimensioned, such as, for example, convex, concave, polygonal, irregular, uniform, non-uniform, staggered, tapered, consistent or variable, depending on the requirements of a particular application.

In one embodiment, tool 70 includes a circuit or integrated circuit device such as, for example, one or more accelerometers, rotary capacitive sensors, solid-state sensors incorporating an accelerometer or a potentiometer, solid-state sensors employing other physical properties (e.g., a magnetic field sensor or other device the employs magneto resistance), or any other mechanical or electronic device that measures an proximity and/or spatial orientation of rod 32 relative to fastener 40.

End 82 is configured to engage display 100. Display 100 includes a screen 118 that displays proximity and/or orientation information of rod 32 relative to fastener 40, obtained from microcontroller 76. Screen 118 may include various types of displays, such as, for example, a cathode ray tube, a light-emitting diode display, a liquid crystal display, an electronic ink, an electroluminescent display, or a plasma display panel. In one embodiment, display 100 shows a “bull's eye” graph, showing the proximity of rod 32 as it approaches tool 70 and fastener 40. Screen 118 may alternatively show an image map created through computed tomography, magnetic resonance, positron emission tomography, ultrasound, or x-ray scans taken prior to the surgical procedure. Preset points may be superimposed onto the image to represent one or more implants, such as, for example, fasteners 40 to which rod 32 is guided during the surgical procedure. In some embodiments, information displayed on screen 118 may be presented in real time during the procedure, can be presented as static images that automatically update periodically upon user commands, or both. In some embodiments, screen 118 may also present static images that display pre-gathered mapping data and do not update.

Display 100 includes buttons 120 to alter display information and system settings such as powering on/off, switching display modes, changing information displayed, displaying battery information, changing display settings such as, for example, screen brightness, calibrating the screen and calibrating the magnetometer. In some embodiments, buttons 120 may include, for example, keys, touchscreens, switches, or knobs.

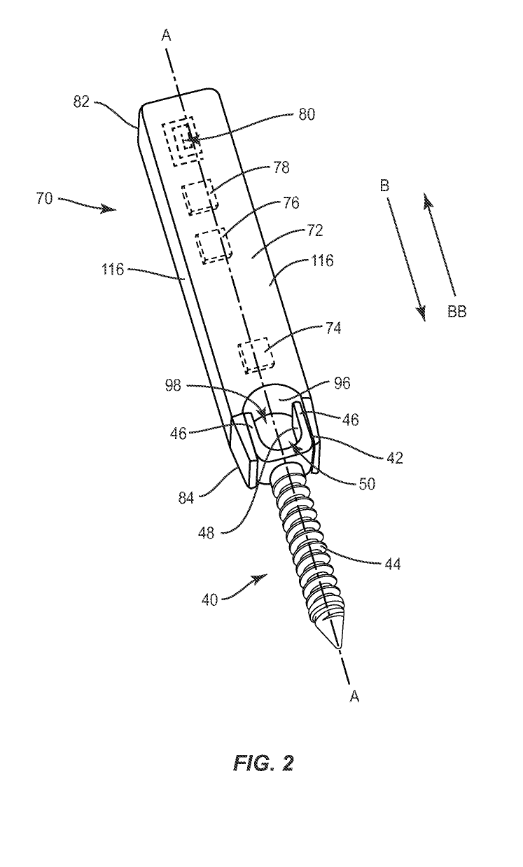

End 82 includes a cylindrical aperture 102 extending parallel to axis A that is configured for alignment with a cylindrical aperture 104 extending parallel to axis A through display 100. A fastener, such as, for example, a set screw 106 is inserted through apertures 102, 104 to engage tool 70 with display. In one embodiment, a distal end of set screw 106 is threaded and is configured to engage a threaded portion of aperture 102 and/or aperture 104. In one embodiment, set screw 106 includes a head 108 having a maximum width that is greater than a maximum width of aperture 102 and/or aperture 104 to prevent head 108 from translating axially through aperture 102 and/or aperture 104 in the direction shown by arrow B in FIG. 2. This configuration allows display 100 to be rotatable relative to tool 70 about axis A, in the direction shown by arrow C in FIG. 2, or, in the direction shown by arrow CC. In some embodiments, display 100 may engage tool 70 alternate fixation configurations, such as, for example, friction fit, pressure fit, locking protrusion/recess, locking keyway and/or adhesive. In some embodiments, aperture 102 and/or aperture 104 may be disposed at alternate orientations relative to axis a, such as, for example, transverse, perpendicular and/or other angular orientations such as acute or obtuse, co-axial and/or may be offset or staggered. In some embodiments, aperture 102 and/or aperture 104 may have alternate cross section configurations, such as, for example, oval, oblong, triangular, square, polygonal, irregular, uniform, non-uniform, offset, staggered, and/or tapered.

In one embodiment, aperture 104 extends through a support arm 110 that is offset from a body 112 of display 100. Body 112 includes a tool engaging portion 114 configured to engage a sidewall 116 of housing 72 when set screw 106 is inserted through apertures 102, 104. This configuration prevents display 100 from rotating relative to tool 70 about axis A, in the direction shown by arrow C in FIG. 1, and, in the direction shown by arrow CC.

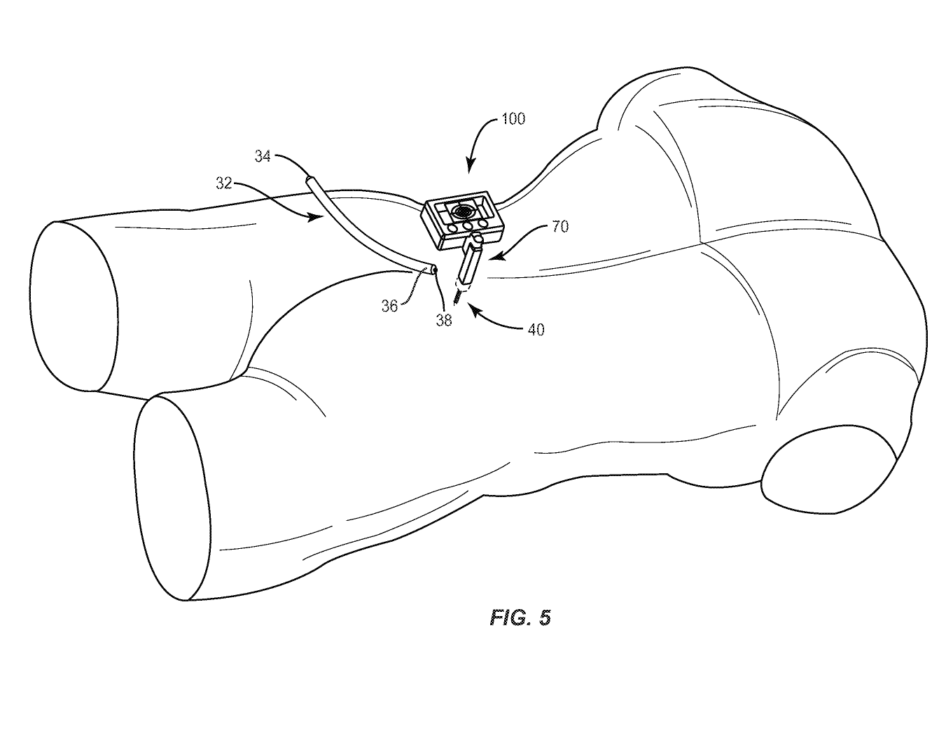

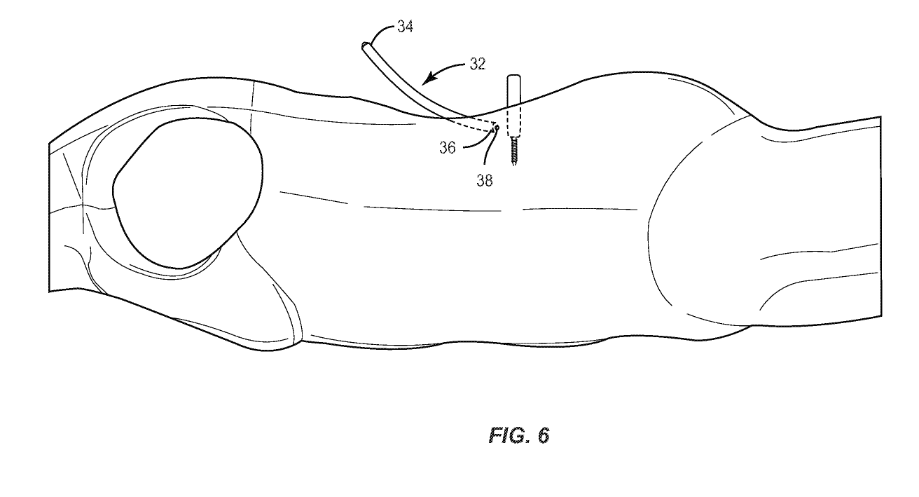

In assembly, operation and use, a spinal implant system, similar to system 30 described above, is employed with a surgical procedure for treatment of a spinal disorder affecting a section of a spine of a patient, as discussed herein. In particular, the spinal implant system is employed with a surgical procedure for treatment of a condition or injury of an affected section of the spine including one or more vertebrae. It is contemplated that a spinal implant system including a fastener 40 may be attached to a vertebra for a surgical arthrodesis procedure, such as fusion, and/or dynamic stabilization application of the affected section of the spine to facilitate healing and therapeutic treatment. Turning now to FIGS. 5 and 6, there is illustrated a method of guiding a first surgical implant, such as, for example, rod 32 relative to a second surgical implant, such as, for example, a fastener 40 for delivering and/or introduction of the implants to adjacent a surgical site using spinal implant system 30, in accordance with the principles of the present disclosure.

In use, to treat the affected section of the spine, a medical practitioner obtains access to a surgical site in any appropriate manner, such as through an incision and retraction of tissues. It is envisioned that the spinal implant system may be used in any existing surgical method or technique including open surgery, mini-open surgery, minimally invasive surgery and percutaneous surgical implantation, whereby one or more vertebrae are accessed through a micro-incision, or sleeve that provide a protected passageway to the area. Once access to the surgical site is obtained, the particular surgical procedure is performed for treating the spinal disorder. A pilot hole is created in a vertebra (not shown) for receiving fastener 40. Fastener 40 is fixed with the vertebra and positioned to receive rod 32 within cavity 50, according to the particular requirements of the surgical treatment.

Tool 70 is positioned relative to fastener 40 such that flanges 92, 94 are aligned with recesses 58, 60. Tool 70 is translated axially, in the direction shown by arrow B in FIG. 2, such that flanges 92, 94 are disposed within recesses 58, 60. The position of tool 70 relative to fastener 40 may be adjusted by translating tool 70 axially, in the direction shown by arrow B, or, in the direction shown by arrow BB. Apertures 102, 104 are aligned with one another and set screw 106 is inserted through apertures 102, 104. Set screw 106 is rotated, in the direction shown by arrow C in FIG. 1, or, in the direction shown by arrow CC, until the threaded portion of set screw 106 engages a threaded portion of aperture 102 and/or aperture 104 to engage display 100 with tool 70. In some embodiments, tool 70 may be engaged with fastener 40 prior to fixation of fastener 40 with the vertebra. In some embodiments, display 100 may be engaged with tool 70 prior to engaging tool 70 with fastener 40.

Magnetometer 74, microcontroller 76 and transmitter 78 are activated with buttons 120. Rod 32 is inserted through the incision and delivered to the surgical site for disposal in cavity 50. As rod 32 is inserted into the incision leading with end 36, magnetometer 74 receives information relating to the position and orientation of magnet 38 disposed with rod 32, relative to magnetometer 74 disposed with fastener 40. The information received by magnetometer 74 is processed by microcontroller 76, which sends a signal to display 100 regarding the position and orientation of magnet 38, disposed with rod 32, relative to magnetometer 74, disposed with fastener 40. The signal is displayed on screen 118 to provide the medical practitioner a visual representation of the position and orientation of magnet 38, disposed with rod 32, relative to magnetometer 74, disposed with fastener 40. Magnetometer 74 is positioned adjacent cavity 50 when tool 70 engages fastener 40 such that the medical practitioner can determine the position and orientation of rod 32 relative to fastener 40 and allows the medical practitioner to selectively adjust the amount and direction rod 32 should be translated relative to fastener 40 to dispose end 36 within cavity 50. The medical practitioner guides rod 32 into channel 98, based upon the visual representation of the position and orientation of magnet 38 relative to magnetometer 74 shown on display 100.

A setscrew may be torqued and threaded with fastener 40 to securely engage rod 32 with fastener 40. Once rod 32 has been guided into cavity 50, tool 70 may be removed from fastener 40. Tool 70 may then be placed on a second bone fastener fixed in a vertebra. Rod 32 is guided to engage the second bone fastener in the same manner as rod 32 was guided to engage fastener 40. Removal and resetting of tool 70 followed by guidance of rod 32 may be repeated to engage other bone fasteners disposed in other vertebrae.

It is contemplated that one or all of the components of spinal implant system 30 may be disposable, peel-pack, pre-packed sterile devices. One or all of the components of spinal implant system 30, for example, tool 70, may be reusable. Spinal implant system 30 may be configured as a kit with multiple sized and configured components.

In some embodiments, the use of microsurgical and image guided technologies may be employed to access, view and repair spinal deterioration or damage, with the aid of spinal implant system 30. Upon completion of the procedure, the surgical instruments, assemblies and non-implant components of spinal implant system 30 are removed from the surgical site and the incision is closed.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal implant guide comprising:
a magnet having a magnetic field and being disposed with a first spinal implant;
a sensor disposed with a first end of a second spinal implant and being configured to measure the magnetic field relative to the second spinal implant; and
indicia representing position and orientation of the first spinal implant relative to the second spinal implant based on the measured magnetic field.

2. A spinal implant guide as recited in claim 1, wherein the sensor includes a magnetometer.

3. A spinal implant guide as recited in claim 1, further comprising a microcontroller for processing the measured magnetic field and generating the indicia.

4. A spinal implant guide as recited in claim 1, wherein the sensor includes a three axis magnetometer.

5. A spinal implant guide as recited in claim 1, further comprising a guidance tool that includes the sensor and a processor for generating the indicia based on the measured magnetic field.

6. A spinal implant as recited in claim 5, wherein the guidance tool is removably disposed with the second spinal implant.

7. A spinal implant guide as recited in claim 1, wherein the magnet is embedded with the first spinal implant.

8. A spinal implant guide as recited in claim 1, wherein the magnet is removably disposed with first spinal implant.

9. A spinal implant guide as recited in claim 1, wherein the indicia includes a visual display disposed with second spinal implant.

10. A spinal implant guide as recited in claim 1, further comprising an antenna disposed with the second spinal implant and communicating with the sensor to transmit the measured magnetic field to a processor.

11. A spinal implant guide as recited in claim 1, wherein the antenna is a wireless interface.

12. A spinal implant guide as recited in claim 1, wherein the measured magnetic field includes changes of strength and spatial orientation of the magnetic field.

13. A spinal implant system comprising:
a first spinal implant extending between a first end and a second end including a magnet having a magnetic field;
a second spinal implant extending between a first end including a sensor and a second end configured to penetrate vertebral tissue, the sensor being configured to measure the magnetic field relative to the second spinal implant;
a processor communicating with the sensor and generating indicia representing position and orientation of the first spinal implant relative to the second spinal implant based on the measured magnetic field; and
a display communicating with the processor.

14. A spinal implant system as recited in claim 13, wherein the sensor includes a magnetometer.

15. A spinal implant system as recited in claim 13, wherein the sensor includes a three axis magnetometer.

16. A spinal implant system as recited in claim 13, wherein the magnet is removably disposed with first spinal implant.

17. A spinal implant system as recited in claim 13, further comprising an antenna disposed with the second spinal implant and communicating with the sensor to transmit the measured magnetic field to a processor.

18. A spinal implant as recited in claim 17, wherein the antenna is a wireless interface.

19. A spinal implant system as recited in claim 13, wherein the measured magnetic field includes changes of strength and spatial orientation of the magnetic field.

20. A spinal implant guide comprising:
a magnet having a magnetic field and being removably disposed with a first spinal implant;
a sensor including a three axis magnetometer disposed with a first end of a second spinal implant, the sensor being configured to measure the magnetic field relative to the second spinal implant the magnetic field including changes of strength and spatial orientation of the magnetic field;
indicia representing position and orientation of the first spinal implant relative to the second spinal implant based on the measured magnetic field, the indicia including a visual display disposed with second spinal implant;
a microcontroller for processing the measured magnetic field and generating the indicia;
an antenna communicating with the sensor to transmit the measured magnetic field to the microcontroller, the antenna comprising a wireless interface; and
a guidance tool removably disposed with the second spinal implant that includes the sensor, the microcontroller and the antenna.
